(19) United States
(12) Patent Application Publication
(10) Pub. No.: US 2009/0035315 A1
(43) Pub. Date: Feb. 5, 2009

(54) Method of Improving Treatments in Rheumatic and Arthritic Diseases

(76) Inventors: Stephan Christgau, Gentofte (DK); Christian Hansen, Vedbaek (DK); Henrik Nilsson, Copenhagen (DK)

Correspondence Address: JONES DAY, 222 EAST 41ST ST, NEW YORK, NY, 10017, US

(21) Appl. No.: 11/629,613

(22) PCT Filed: Jun. 17, 2005
(86) PCT No.: PCT/DK05/00404

§ 371 Date: Mar. 10, 2008

(30) Foreign Application Priority Data
Jun. 17, 2004 (DK) PA 2004 00950

(51) Int. Cl. A61K 39/395; A61K 31/662; A61K 31/351; A61K 31/715; A61K 31/724; A61K 31/192; A61K 31/18; A61K 31/167; A61K 31/60; A61K 31/235; A61K 31/5415; A61K 31/56; A61K 38/00; A61K 31/415

(52) U.S. Cl. 424/145.1; 514/108; 514/459; 514/406; 514/21; 514/54; 514/171; 514/58; 514/226.5; 514/569; 514/570; 514/533; 514/605; 514/159; 514/629

(57) ABSTRACT

Improved treatments of joint diseases, such as, e.g. osteoarthritis and rheumatoid arthritis, and pain, wherein a strontium-containing compound is administered alone or in combination with one or more second therapeutically and/or prophylactically active substances, selected from the group consisting of bisphosphonates, glucosamine, pallitative agents, analgesic agents, disease modifying anti-rheumatic compounds (DMARDs), selective estrogen receptor modulators (SERMs), aromatase inhibitors, non-steroidal anti-inflammatory agents (NSAIDs), COX-2 inhibitors, COX-3 inhibitors, opioids, inhibitors/antagonists of IL-1, inhibitors/antagonists of TNF-alpha, inhibitors of matrix metallo-proteinases (MMPs), cathepsin K inhibitors, inhibitors/antagonists of RANK-ligand, statins, glucocorticoids, chondroitin sulphate, NMDA receptor antagonists, inhibitors of interleukin-I converting enzyme, Calcitonin gene related peptide antagonists, glycine antagonists, vanilloid receptor antagonists, inhibitors of inducible nitric oxide synthetase (iNOS), N-acetylcholine receptor agonists, neurokinin antagonists, neuroleptic agents, PAR2 receptor antagonists and anabolic growth factors acting on joint tissue components. Pharmaceutical compositions comprising a strontium-containing compound and a second therapeutically and/or prophylactically active substance as defined above.

### FIELD OF THE INVENTION

The present invention relates to improved treatments of osteoarthritis, rheumatoid arthritis and pain, wherein a strontium-containing compound is administered alone or in combination with a second therapeutically and/or prophylactically active substance.

### BACKGROUND OF THE INVENTION

Osteoarthritis (OA), which is also called “degenerative joint disease” or arthrosis, is one of the most common disorders of the musco-skeletal system. The World Health Organization ranks OA the fourth most serious health problem in women and the eighth most serious in men, when measured by disability-adjusted life years. The most common joints affected by OA are the knees, hands, hips and big toes. The exact causes of this condition are unknown and difficult to resolve as multiple factors play a role in the initiation and progression of the disease. It is associated with a certain genetic background as individuals who have a history of OA in their family have increased risk of developing the disease, but also many other factors play a role as exemplified by the susceptibility of people who have previously had a serious injury, such as ligament or meniscal damage, or certain forms of joint-surgery to develop OA.

The prevalence of OA increases with age and it is a prominent cause of disability and poor quality of life among the elderly. Among subjects older than 60-70 years of age practically all will present some forms of articular changes and deterioration with the characteristics of osteoarthritis, although not all changes will manifest itself in the symptoms of pain and reduced mobility associated with more advanced stages of the disease. The prevalence of OA is higher in women than in men, and in women the incidence of OA increases significantly after the menopause. However, in older subjects (i.e. individuals above 75 years of age) OA prevalence is so common among both men and women that no significant gender differences can be demonstrated.

A central element of the disease process in OA is the non-reversible degradation of articular cartilage, which starts prior to clinical diagnosis of OA and persists until the end stage of the disease where most articular cartilage of affected joints is lost. At this stage the mobility of the joint is severely compromised, and joint replacement surgery remains the only treatment option. In OA joints certain other macroscopic abnormalities such as cartilage degeneracy, trabecular architectural deterioration and osteophytes (small abnormal bone outgrowths in the rims of the bone ends at affected joints) occur and develop on the stripped part of the subchondral bone.

Symptoms of OA are pain, swelling and stiffness of the articulation. This disease process in OA is likely to be initiated many years prior to clinical diagnosis of the disease, and it persists until the end stage of the disease where almost all articular cartilage of the affected joints is lost. At this stage the mobility of the joint is severely compromised, and joint replacement surgery remains the only treatment option. Due to this prolonged development of the pathological joint changes associated with OA it is very difficult to elucidate the factors involved in the early phases of the disease process.

The most common treatments in clinical use for the management of OA are focused on the symptoms of the disease rather than the underlying structural damage of the joint tissues. Thus, the therapeutic options presented to the patient today are mainly analgesics and other palliative agents such as non-steroidal anti inflammatory drugs (NSAIDs).

Rheumatoid arthritis (RA) is an inflammatory condition where articular cartilage of affected joints is being degraded by an active process involving cells of the immune system as well as the tissues of the joint (i.e. the synovial membrane, the cartilage and subchondral bone). The etiology of RA is complex and a number of environmental and genetic factors have been suggested a role in the development of the disease.

However, as a significant elevation in both systemic and local signaling molecules and other active components of immune system activation can be detected in this disease it is clearly an immune-system related disorder. Among the characteristic hallmarks of the disease is the presence of cartilage destruction, bone erosions, periarticular osteoporosis and generalized bone loss resulting in increased prevalence of osteoporotic fractures. Some of the disease mechanisms responsible for focal bone loss may be similar to processes of generalized osteoporosis and associated with osteoclast activation. Generalized bone loss in patients with RA will occur as a result of the systemic and local activation of the immune-system and the presence of proinflamatory cytokines with an ability to stimulate resorption of bone. In turn these mediators accelerate bone turnover and systemic bone loss. Recent research demonstrates that many of the proinflammatory cytokines may directly mediate an elevation in osteoclast activation. In addition, bone loss also takes place focally as a consequence of the arthritic disease process. Another significant contributing factor to systemic bone loss in RA is the common therapeutic use of glucocorticoids, which in addition to their well-documented anti-inflammatory effect also have a stimulating effect on bone resorption.

RA is more prevalent in woman. The disease is not well correlated with age and it may occur in all age groups from juveniles until the oldest age Just as estrogen is likely to have a chondroprotective potential in OA management there is also reports of a similar therapeutic effect in RA (Forsblad d'Elia et al Arthritis Res Ther. 2004; 6(5):457-68). A subgroup of the disease called juvenile RA has been described, but at present it is unclear whether this represents a truly different etiology/pathology compared to the ‘conventional’ adult onset RA.

Also in RA the major clinical manifestation of the disease is the presence of swollen and tender joints with significant pain both systemically and localized in the affected joints. Thus, analgesic and palliative treatments remains one of the most used therapeutic interventions used in the clinical management of RA.

Studies in humans and animal models of OA an RA have demonstrated a progressive depletion of articular cartilage matrix macromolecules as the disease develops. In RA the cartilage degradation tends to occur more rapidly due to the potent catabolic stimuli evoked by the active inflammatory response and immune system components mediating the tissue destruction in the disease. The progression of joint destruction varies widely between individual patients with a marked cyclical pattern characterized by periods of elevated disease activity (flare ups) intermittent with more ‘silent’ periods. This cyclical pattern of disease activity is especially prominent for RA.

The most commonly used drugs for the treatment of RA are NSAIDs and Opioids used for treating the pain and symptoms of the patients and disease modifying anti-rheumatic drugs (DMARD's) and corticosteroids as well as more specific anti-inflammatory agents such as TNF-αt or IL-1 antagonists. In OA treatment, NSAID and DMARD's also play an important role. The use of NSAIDs and simple analgesics e.g. paracetamol, have been shown to reduce the pain of OA. In addition topical NSAIDs can provide some pain relief and are associated with fewer side effects than the systemic drug treatments. Intra-articular steroid injections can be used for inflammatory flares, but in established OA the effects are short-lived, In RA better effects have been obtained with systemic as well as intra-articular steroid administration, and this remains one of the most common treatment options for the disease, in spite of the adverse effects associated with long term steroid use such as accelerated systemic bone loss leading to osteoporosis and an increased risk of fragility fracture. In more advanced cases of OA, hip and knee replacements are an effective surgical option for relieving pain and improving function.

The aim of current therapies of these diseases is mainly to relieve pain and disease symptoms. NSAID, opioids and DMARD's have proved effectiveness in relieving the symptoms of OA and RA but their effect on decreasing cartilage catabolism has not been well documented. Some of them, like sodium salicylate, have shown inhibiting properties of the proteoglycan synthesis which may jeopardize the cartilage repair process. Other drugs, such as tiaprofenic acid, which do not inhibit the proteoglycan synthesis, have shown in vitro that they are able to decrease OA cartilage catabolism, (Pelletier et al. The Journal of Rheumatology 1989; 16:5, 646-655). However they have been unable to provide any significant protective effect in development of OA when administrated to patients suffering from the latter, (Edward C. Huskisson et al. J Rheumatol 1995; 22:10-1941-1946). Doxycycline, a member of the tetracycline family, was also shown to reduce, in vivo, the severity of OA lesions in the dog anterior cruciate ligament trans-section (ACL) model of joint damage induced OA while reducing metalloprotease activity, (Yu et al. Arthr Rheum 35:1150-1159, 1992). Recent data suggests that the action of corticosteroids is associated with a reduction in the synthesis of the cartilage matrix degrading MMP, stromelysin-1 by chondrocytes. (see: Pelletier et al., J Arthr Rheum 37:414-423, 1994; and Pelletier et al., J Lab Invest 72:578-586, 1995).

In the clinical management of OA, the focus of medical intervention has been to relieve disease symptoms (i.e. by using Non-Steroidal-Anti-Inflammatory Drugs (NSAID) and the newer COX-2 inhibitors). None of the drugs in current clinical use, with the exception of glucosamine sulphate has demonstrated significant effects to halt the underlying tissue destruction (i.e. articular cartilage thinning and subchondral bone changes) (Christgau et al. Clin Exp Rheumatol. 2004; 22: 36-42). It is very questionable if the palliative agents used in the clinical management of OA have any structure modifying effects. In fact, recent reviews of the literature indicates that different classes of NSAIDs may have effects on chondrocytes ranging from deleterious to beneficial with regard to glycosaminoglycan synthesis. Very few, if any, therapies are available that has a convincing effect of slowing or halting the underlying cartilage degradation, which is the prime culprit causing the progressive joint destruction accompanying the disease. Thus, there is an unmet therapeutic need for compounds, which can act on the cells and enzyme systems mediating the cartilage degradation in OA.

In the management of acute and chronic pain in joint diseases such as RA and OA, the ability to prevent the onset of pain, lessen its intensity, and interfere with the development of sensitization contributing to hyperalgesia for days following traumatic pain can greatly benefit the patient as pain represents the main clinical symptoms. Accordingly the palliative treatment is important and effective management of the joint diseases. In situations where pain can be anticipated, i.e. at the early clinical signs of a flare up in disease activity, the NSAID may be optimized by administration of elevated doses and continuing to dose the NSAID on a regular schedule to minimize pain and inflammation. Patients benefit from receiving optimal NSAID doses, and in some cases very high doses of these palliative agents are required to efficiently relieve the pain. In conditions of chronic pain, the dosing of palliative agents are of paramount importance, and as RA and OA patients are likely to receive the drugs over long periods of time due to the chronic nature of the diseases, the side effects of these interventions becomes of paramount importance. This represents a major problem in current clinical practice as most NSAID's and other analgesic agents are associated with severe gastro intestinal side effects.

### SUMMARY OF THE INVENTION

In one aspect, the invention relates to a pharmaceutical composition comprising i) a strontium-containing compound as a first therapeutically and/or prophylactically active substance, and ii) a second therapeutically and/or prophylactically active substance selected from the group consisting of bisphosphonates, glucosamine, palliative agents, analgesic agents, disease modifying anti-rheumatic compounds (DMARDs), selective estrogen receptor modulators (SERMs), aromatase inhibitors, non-steroidal anti-inflammatory agents (NSAIDs), COX-2 inhibitors, COX-3 inhibitors, opioids, inhibitors/antagonists of IL-1, inhibitors/antagonists of TNF-α, inhibitors of matrix metallo-proteinases (MMPs), cathepsin K inhibitors, inhibitors/antagonists of RANK-ligand, statins, glucocorticoids, chondroitin sulphate, NMDA receptor antagonists, inhibitors of interleukin-I converting enzyme, Calcitonin gene related peptide antagonists, glycine antagonists, vanilloid receptor antagonists, inhibitors of inducible nitric oxide synthetase (iNOS), N-acetylcholine receptor agonists, neurokinin antagonists, neuroleptic agents, PAR2 receptor antagonists and anabolic growth factors acting on joint tissue components.

In some embodiments of the pharmaceutical composition, the second therapeutically and/or prophylactically active substance is a bisphosphonate or a glucosamine. For instance, in specific embodiments the second therapeutically and/or prophylactically active substance is a bisphosphonate selected from the group consisting of ibandronate, zoledronate, alendronate, risedronate, ethidronate, chlodronate, tiludronate, minodronate, incadronate, olpadronate and pamidronate.

In some embodiments of the pharmaceutical composition, the second therapeutically and/or prophylactically active substance is glucosamine sulphate.

In other embodiments of the pharmaceutical composition, the second therapeutically and/or prophylactically active substance is selected from the group consisting of heroin, fentanyl, morphine, oxycodone, hydrocodone, methadone, buprenorphine, pentazocine, butorphanol, dezocine, nalbuphine, meperidine, normeperidine, hydromorphone, codeine, levorphanol, tramadol, endorphin, nociceptin, endomorphin, and active metabolites thereof.

In other embodiments of the pharmaceutical composition, the second therapeutically and/or prophylactically active substance is an NSAID selected from the group consisting enolic acis such as piroxicam, tenoxicam and meloxicam, heteroaryl acetic acids such as diclofenac, tolmetin, ketorolac, misoprostol and zomepirac; Indole and indene acetic acids such as indomethacin, mefenamic acid, sulindac and etodolac; Para-amino phenol derivates such as phenacetin and acetaminophen; propionic acids including naproxen, flurbiprofen, fenoprofen, oxaprozin, carprofen, ketoprofen and ibuprofen; Sulphonanilides such as Nimesulide; fenamates including mefenamic acid, meclofenamate and flufenamic acid; alkanones such as nabumetome; pyrazolones including phenylbutazone, oxyphenbutazone, antipyrine, aminopyrine and kebuzone, salicylates including acetyl salicylate (aspirin), salicylate, salsalate, difunisal, olsalazine, fendosal, sulfasalazine and thiosalicylate; paracetamol; or a pharmaceutically acceptable salt thereof.

In other embodiments of the pharmaceutical composition, the second therapeutically and/or prophylactically active substance is a selective COX-2 inhibitor, that has a 10 fold higher affinity for the COX-2 isoenzyme compared to COX-1, the selective COX-2 inhibitor being selected from the group consisting of rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-1-hydroxy-6,6dimethyl-6H-dibenzo[b,d]pyran carboxylic acid (CT-3), 2(5H)-Furanone, 5,5-dimethyl (1-methylethoxy) [4(methylsulfonyl)phenyl]-(DFP); Carprofen (RIMADYLO), (Acetyloxy)-benzoic acid, 3-[(nitrooxy)methyl]phenyl ester (NCX4016), P54 (CAS Reg. No. 130996 0) 2,6-Bis(1,1-dimethylethyl) [(E)-(2-ethyl-1,1-dioxo isothiazolidinylidene)methyl]phenol (S-2474), 5(R)-Thio sulfonamide-3(2H)-benzofuranone (SVT-2016) and N-[3-(Formyl-amino)oxo phenoxy-4H benzopyran yl]methanesulfonamide (“T-614”); or a pharmaceutically acceptable salt thereof.

In other embodiments of the pharmaceutical composition, the second therapeutically and/or prophylactically active substance is a DMARD selected from the group consisting of doxycycline, chondroitin sulfate, methotrexate, leflounomide (ARAVA®, Aventis), dimethylnitrosamine, azatriopine, hydroxychloroqine, cyclosporine, minocycline, salazopyrine, penicillamine, aurothiomalate (gold salt), cyclophosphamide, azathioprine and pharmacologically active metabolites thereof.

In other embodiments of the pharmaceutical composition, the second therapeutically and/or prophylactically active substance is a selective estrogen receptor modulator selected from the group consisting of raloxifene, arzoxifene, droloxifene, tamoxifen, 4-hydroxy-tamoxifen, 4′-iodotamoxifen, toremifene, (deaminohydroxy)-toremifene, chlomiphene, levormeloxifene, ormeloxifene, chroman derivatives, coumarin derivatives, idoxifene, nafoxidine, TAT-59, LY-353381, CP-336156, MDL-103323, EM-800, ICI-182, ICI 183,780, ICI 164,384, ICI 183,780, ICI 164,384, diethylstilbesterol, genistein, nafoxidine, nitromifene, moxesterol, diphenol hydrochrysene, erythro-MEA, allenolic acid, equilin-3-sulphate, cyclophenyl, chlorotrianisene, ethamoxytriphetol, lasofoxifene, bazedoxifene, genistein, tibolone, ospemifene, tesmilifene, droloxifene, panomifene, zindoxifene, meproxifene and faslodex as well as pharmacologically active metabolites of any of the listed substances.

In other embodiments of the pharmaceutical composition, the second therapeutically and/or prophylactically active substance is selected from the group consisting of inhibitors of IL-1, such as anakinra, a monoclonal antibody against IL-1 and soluble IL-1 receptor derivatives, including derivatives modified by attachment of polyethylene glycol.

In other embodiments of the pharmaceutical composition, the second therapeutically and/or prophylactically active substance is an inhibitor of interleukin-I converting enzyme (ICE), such as Pralnacasan.

In other embodiments of the pharmaceutical composition, the second therapeutically and/or prophylactically active substance is selected from the group consisting of inhibitors of TNF-α including etanercept (Enbrel®,), adalimumab (Humira®), and infliximab (Remicade®).

In other embodiments of the pharmaceutical composition, the second therapeutically and/or prophylactically active substance is selected from the group consisting of OPG and other inhibitors of RANK-ligand including monoclonal antibody AMG-162.

In other embodiments of the pharmaceutical composition, the second therapeutically and/or prophylactically active substance is selected from the group consisting of inhibitors of a matrix metalloproteinase inhibitor including inhibitors of aggrecanase, MMP-1, MMP-13, MMP-3, cathepsin K and another protease involved in the catabolic processes of tissue destruction in joint diseases such as OA and RA.

In other embodiments of the pharmaceutical composition, the second therapeutically and/or prophylactically active substance is chondroitin sulphate or keratin sulphate.

In other embodiments of the pharmaceutical composition, the second therapeutically and/or prophylactically active substance is hyaluronic acid (including for inter-articular injection).

In other embodiments of the pharmaceutical composition, the second therapeutically and/or prophylactically active substance is a glucocorticoid such as prednisolone, prednisone, methylprednisolone, betamethasone, hydrocortisone, cortisone, triamcinolone, dexamethasone, beclomethasone, budesonide, deoxycortone or fludrocortisone.

In other embodiments of the pharmaceutical composition, the second therapeutically and/or prophylactically active substance is an endothelin-1 antagonist/inhibitor.

In other embodiments of the pharmaceutical composition, the second therapeutically and/or prophylactically active substance is an anabolic growth factor derived from bone or cartilage matrix proteins such as segments of or fragments from collagen type I, collagen type II, collagen type IX, collagen type XI, bone sialo protein (BSP), osteonection, osteopontin, osteocalcin (also known as bone GLA protein), cartilage oligomeric matrix protein (COMP), cartilage intermediate layer protein (CILP) and aggrecan.

In other embodiments of the pharmaceutical composition, the second therapeutically and/or prophylactically active substance is an anabolic growth factor such as human growth hormone (hGH), parathyroid hormone (PTH), glucagon like peptide-2 (GLP-2), Insulin like growth factor-1 (IGF-1) with or without IGF binding protein 3 (IGFBP-3).

In other embodiments of the pharmaceutical composition, the second therapeutically and/or prophylactically active substance is a statin such as nystatin pravastatin, fluvostatin, atorvastatin and cerivastatin and therapeutically active derivatives thereof.

In other embodiments of the pharmaceutical composition, the second therapeutically and/or prophylactically active substance is an aromatase inhibitor.

In other embodiments of the pharmaceutical composition, the second therapeutically and/or prophylactically active substance is a sulphated cyclodextrin.

In some embodiments of the pharmaceutical composition, the strontium-containing compound is selected from the group of organic strontium salts comprising: strontium malonate, strontium succinate, strontium fumarate, strontium ascorbate, strontium aspartate in either L and/or D-form, strontium glutamate in either L- and/or D-form, strontium pyruvate, strontium tartrate, strontium glutarate, strontium maleate, strontium methanesulfonate, strontium benzenesulfonate and strontium ranelate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-Iminopiperidine, strontium methotrexate, strontium salsalate and strontium sulfasalazinate.

In certain embodiments of the pharmaceutical composition, the strontium-containing compound and the second therapeutically and/or prophylactically active substance are present in a single composition.

In some embodiments of the pharmaceutical composition, the strontium-containing compound and the second therapeutically and/or prophylactically active substance are present in a kit comprising a first and a second container, the first container comprising the strontium-containing compound and the second container comprising the second therapeutically and/or prophylactically active substance. In specific embodiments, the pharmaceutical composition further comprises instructions for simultaneous or sequential use of the first and the second therapeutically and/or prophylactically active substance.

In certain embodiments of the pharmaceutical composition, the pharmaceutical composition is designed for oral administration.

In another aspect, the invention relates to the use of a strontium-containing compound for the preparation of a pharmaceutical composition for the treatment of a joint disease such as OA and RA.

In another aspect, the invention relates to the use of a combination of i) a strontium-containing compound and ii) a second therapeutically and/or prophylactically active substance selected from the group consisting of bisphosphonates, glucosamine, palliative agents, analgesic agents, disease modifying anti-rheumatic compounds (DMARDs), selective estrogen receptor modulators (SERMs), aromatase inhibitors, non-steroidal anti-inflammatory agents (NSAIDs), COX-2 inhibitors, COX-3 inhibitors, opioids, inhibitors/antagonists of IL-1, inhibitors/antagonists of TNF-α, inhibitors of matrix metallo-proteinases (MMPs), cathepsin K inhibitors, inhibitors/antagonists of RANK-ligand, statins, glucocorticoids, chondroitin sulphate, NMDA receptor antagonists, inhibitors of interleukin-I converting enzyme, Calcitonin gene related peptide antagonists, glycine antagonists, vanilloid receptor antagonists, inhibitors of inducible nitric oxide synthetase (iNOS), N-acetylcholine receptor agonists, neurokinin antagonists, neuroleptic agents, PAR2 receptor antagonists and anabolic growth factors acting on joint tissue components for the preparation of a pharmaceutical composition as defined above for the treatment of a joint disease such as OA and RA.

In some embodiments of this use, the second therapeutically and/or prophylactically active substance is a bisphosphonate or a glucosamine.

In other embodiments of this use, the second therapeutically and/or prophylactically active substance is selected from the group consisting of palliative agents, analgesic agents and anti-inflammatory agents.

In another aspect, the invention relates to the use of a combination of i) a strontium-containing compound and ii) a second therapeutically and/or prophylactically active substance selected from the group consisting of bisphosphonates, glucosamine, palliative agents, analgesic agents, disease modifying anti-rheumatic compounds (DMARDs), selective estrogen receptor modulators (SERMs), aromatase inhibitors, non-steroidal anti-inflammatory agents (NSAIDs), COX-2 inhibitors, COX-3 inhibitors, opioids, inhibitors/antagonists of IL-1, inhibitors/antagonists of TNF-α, inhibitors of matrix metallo-proteinases (MMPs), cathepsin K inhibitors, inhibitors/antagonists of RANK-ligand, statins, glucocorticoids, chondroitin sulphate, NMDA receptor antagonists, inhibitors of interleukin-I converting enzyme, Calcitonin gene related peptide antagonists, glycine antagonists, vanilloid receptor antagonists, inhibitors of inducible nitric oxide synthetase (iNOS), N-acetylcholine receptor agonists, neurokinin antagonists, neuroleptic agents, PAR2 receptor antagonists and anabolic growth factors acting on joint tissue components for the preparation of a pharmaceutical composition as defined above for alleviating pain.

In some embodiments of this use, the second therapeutically and/or prophylactically active substance is selected from the group consisting of palliative agents, analgesic agents and anti-inflammatory agents.

In certain embodiments of this use, the pain is selected from the group consisting of: osteoarthritic pain, rheumatoid arthritic pain, juvenile chronic arthritis associated pain, juvenile idiopathic arthritis associated pain, Spondyloarthropathies (such as ankylosing spondylitis (Mb Bechterew) and reactive arthritis (Reiter's syndrome)) associated pain, pain associated with psoriatic arthritis, gout pain, pain associated with pseudogout (pyrophosphate arthritis), pain associated with systemic lupus erythematosus (SLE), pain associated with systemic sclerosis (scleroderma), pain associated with Behçet's disease, pain associated with relapsing polychondritis, pain associated with adult Still's disease, pain associated with transient regional osteoporosis, pain associated with neuropathic arthropathy, pain associated with sarcoidosis, arthritic pain, rheumatic pain, joint pain, osteoarthritic joint pain, rheumatoid arthritic joint pain, juvenile chronic arthritis associated joint pain, juvenile idiopathic arthritis associated joint pain, Spondyloarthropathies (such as ankylosing spondylitis (Mb Bechterew) and reactive arthritis (Reiter's syndrome)) associated joint pain, joint pain associated with psoriatic arthritis, gout joint pain, joint pain associated with pseudogout (pyrophosphate arthritis), joint pain associated with systemic lupus erythematosus (SLE), joint pain associated with systemic sclerosis (scleroderma), joint pain associated with Behçet's disease, joint pain associated with relapsing polychondritis, joint pain associated with adult Still's disease, joint pain associated with transient regional osteoporosis, joint pain associated with neuropathic arthropathy, joint pain associated with sarcoidosis, arthritic joint pain, rheumatic joint pain, acute pain, acute joint pain, chronic pain, chronic joint pain, inflammatory pain, inflammatory joint pain, mechanical pain, mechanical joint pain, pain associated with the fibromyalgia syndrome (FMS), pain associated with polymyalgia rheumatica, monarticular joint pain, polyarticular joint pain, nociceptiv pain, neuropathic pain, psychogenous pain, pain of unknown etiology, and pain mediated by IL-6, IL-6 soluble receptor, or IL-6 receptor.

In some embodiments of the use, the pain is other than joint pain, osteoarthritic pain, rheumatoid arthritic pain, and inflammatory joint pain, and the pain is mediated by IL-6, IL-6 soluble receptor, or IL-6 receptor.

In certain embodiments of the use, the pain is mediated by a protein or protein and its receptor selected from: oncostatin-M, oncostatin-M and oncostatin-M receptor, leukemia inhibitor factor (“LIF), LIF and leukemia inhibitor factor receptor (“LIF-R”), interleukin-1 (“IL-1”), and interleukin-1 receptor (“IL1-R”).

In some embodiments of the use, the pain is other than joint pain, osteoarthritic pain, rheumatoid arthritic pain, and inflammatory joint pain, and the pain is mediated by endothelin.

In certain embodiments of the use, the pain is associated with a surgical procedure in a patient with a clinical diagnosis of OA.

In another aspect, the invention relates to a method for the treatment of a joint disease selected among OA and RA, the method comprising administering to a subject including an animal such as a human in need thereof an effective dose of a strontium-containing compound via the oral route.

In still another aspect, the invention relates to a method for the treatment of a joint disease selected among OA and RA, the method comprising administering to a subject including an animal such as a human in need thereof an effective dose of a strontium-containing compound via the oral route and an effective dose of a second therapeutically and/or prophylactically active substance selected from the group consisting of bisphosphonates, glucosamine, palliative agents, analgesic agents, disease modifying anti-rheumatic compounds (DMARDs), selective estrogen receptor modulators (SERMs), aromatase inhibitors, non-steroidal anti-inflammatory agents (NSAIDs), COX-2 inhibitors, COX-3 inhibitors, opioids, inhibitors/antagonists of IL-1, inhibitors/antagonists of TNF-α, inhibitors of matrix metallo-proteinases (MMPs), cathepsin K inhibitors, inhibitors/antagonists of RANK-ligand, statins, glucocorticoids, chondroitin sulphate, NMDA receptor antagonists, inhibitors of interleukin-I converting enzyme, Calcitonin gene related peptide antagonists, glycine antagonists, vanilloid receptor antagonists, inhibitors of inducible nitric oxide synthetase (iNOS), N-acetylcholine receptor agonists, neurokinin antagonists, neuroleptic agents, PAR2 receptor antagonists and anabolic growth factors acting on joint tissue components.

In another aspect, the invention relates to a method for the treatment of a joint disease selected among OA and RA, the method comprising administering to a subject including an animal such as a human in need thereof an effective dose of a strontium-containing compound via the oral route and an effective dose of a second therapeutically and/or prophylactically active substance selected from the group consisting of palliative agents, analgesic agents and anti-inflammatory agents.

In some embodiments of these methods, the methods are for alleviating pain in a subject suffering from a joint disease selected from OA and RA.

In some embodiments of these methods, the strontium-containing compound is selected from the group of organic strontium salts comprising: strontium malonate, strontium succinate, strontium fumarate, strontium ascorbate, strontium aspartate in either L and/or D-form, strontium glutamate in either L- and/or D-form, strontium pyruvate, strontium tartrate, strontium glutarate, strontium maleate, strontium methanesulfonate, strontium benzenesulfonate and strontium ranelate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-Iminopiperidine, strontium methotrexate, strontium salsalate and strontium sulfasalazinate.

In some embodiments of these methods, the second therapeutically and/or prophylactically active substance is as defined above.

In certain embodiments of these methods, the strontium-containing compound and the second therapeutically and/or prophylactically active substance are administered in the form of a pharmaceutical composition wherein the strontium-containing compound and the second therapeutically and/or prophylactically active substance are present in a single composition (e.g., for oral administration); or the strontium-containing compound and the second therapeutically and/or prophylactically active substance are present in a kit (e.g., for oral administration) comprising a first and a second container, the first container comprising the strontium-containing compound and the second container comprising the second therapeutically and/or prophylactically active substance. The kit optionally comprises instructions for simultaneous or sequential use of the first and the second therapeutically and/or prophylactically active substance.

In some embodiments of these methods, the joint disease is OA and the subject is given a daily dose of ionic strontium corresponding to 100-2000 mg.

In other embodiments of these methods, the joint disease is RA and the subject is given a daily dose of ionic strontium corresponding to 100-2000 mg.

In some embodiments of these methods, a strontium compound is given in a daily dose of ionic strontium corresponding to 100-2000 mg together with meloxicam administered in a daily dose of 5-20 mg.

In other embodiments of these methods, a strontium compound is given in a daily dose of ionic strontium corresponding to 100-2000 mg together with piroxicam administered in a daily dose of 10-30 mg.

In other embodiments of these methods, a strontium compound is given in a daily dose of ionic strontium corresponding to 100-2000 mg together with naproxen administered in a daily dose of 500-1500 mg.

In other embodiments of these methods, a strontium compound is given in a daily dose of ionic strontium corresponding to 100-2000 mg together with dexibuprofen administered in a daily dose of 500-1600 mg.

In other embodiments of these methods, a strontium compound is given in a daily dose of ionic strontium corresponding to 100-2000 mg together with ibuprofen administered in a daily dose of 1000-3200 mg.

In other embodiments of these methods, a strontium compound is given in a daily dose of ionic strontium corresponding to 100-2000 mg together with celecoxib administered in a daily dose of 100-200 mg.

In other embodiments of these methods, a strontium compound is given in a daily dose of ionic strontium corresponding to 100-2000 mg together with salsalate administered in a daily dose of 1000-3000 mg.

### DESCRIPTION OF THE INVENTION

It has previously been suggested that ionic non-radioactive strontium may have beneficial effects on synthesis of proteoglycans and collagen type II by chondrocytes residing within the extracellular matrix of articular cartilage and responsible for the turnover of the organic matrix of the tissue, i.e. that certain strontium compounds may have an anabolic effect on cartilage. It is also known that stable strontium acts on bone turnover by reducing bone resorption while maintaining or even increasing bone formation (J Reginster, Curr. Pharm. Design, 8: 1907-1916, 2002). These findings have been coupled to a potential use of one specific strontium salt in the treatment of OA as disclosed in EP0813 869B1.

It has also been disclosed in WO 03028742 that certain strontium containing compounds may find use in treatment of soft tissue pain, particularly relevant for topical administration. However this source does not disclose anything about the specific anti catabolic effects of ionic strontium on cartilage metabolism, which is of particular relevance for the present invention.

We have discovered that compounds containing ionic non-radioactive stable strontium have a significant palliative as well as anti-catabolic effects when administered orally to a subject in need thereof. Importantly strontium-containing compounds can exert their palliative effect by mechanism distinct from the cellular processes targeted by the existing therapeutic agents for pain treatment and prevention such as NSAID's or opioids. The present invention therefore provides a new method for the treatment, prevention or alleviation of pain comprising the administration by the oral route of one or more strontium-containing compounds either alone or in combination with one or more analgesic and/or palliative and/or structure modifying agents such as NSAIDs, DMARDs, opioids, COX-2 inhibitors, inhibitors of TNF-α, inhibitors of IL-1, leptin antagonists, inhibitors of substance P, inhibitors of matrix metallo-proteinases (MMPs), inhibitors/antagonists of RANK-ligand, glucocorticoids, glucosamine, chondroitin sulphate, hyaluronic acid and anabolic growth factors acting on joint tissue components such as endothelin-1, IGF-1 and vascular endothelial growth factor (VEGF).

Of further relevance for the present invention we have found that not only does strontium possess a putative anabolic effect on cartilage matrix synthesis, but it also evokes an anti-catabolic effect whereby it decreases the degradation of the cartilage matrix. This may be the most important mechanism by which strontium can exert a chondroprotective or structure modifying effect of therapeutic relevance for the prophylaxis and treatment of diseases such as OA and RA, but the general properties of strontium with respect to interactions with subchondral bone and articular cartilage is equally well suited for the medical intervention in joint diseases. The anti-catabolic effect of strontium on cartilage turnover is exerted when the compound is administered alone or in combination with other therapeutic agents and thus there is a substantial therapeutic potential in using one or more strontium containing compounds in combination with other pharmaceutical products. This dual action of strontium, combined with the relatively mild antiresorptive and proanabolic effects on bone tissue, makes strontium optimal for the use in therapy of joint diseases where aberrant regulation of both bone and cartilage tissue is involved in the disease pathology.

Of further interest, we have found that strontium is also able to exert a palliative effect of relevance to the pain and symptoms associated with joint diseases characterized by local/articular and/or systemic elevation in inflammation. In particular strontium is able to evoke this effect in synergy with current analgesic or palliative agents in use in the clinical practice today for the treatment of RA and OA. This means that a combination therapy comprising a strontium component, such as a strontium salt, and a palliative and/or analgesic and/or disease modifying and/or anti-inflammatory agent such as an NSAID, opioid, steroid, glucocorticoid, DMARD, COX-2 Inhibitor, inhibitors of matrix metallo-proteinases (MMPs), inhibitors/antagonists of RANK-ligand, leptin antagonists, glucocorticoids, glucosamine including glucosamine sulphate, chondroitin sulphate, hyaluronic acid and anabolic growth factors acting on joint tissue components such as endothelin-1, IGF-1 and vascular endothelial growth factor (VEGF) or others is especially well suited for the clinical management of diseases where systemic and/or local inflammation is elevated and catabolic destructive processes of bone and cartilage occur, such as OA and RA. Examples of suitable strontium salts are given below and examples of suitable NSAIDs, opioids, steroids, DMARDs, COX-2 inhibitors etc. for use in combination with a strontium-containing compound according to the invention are given under the heading “Definitions”.

The dosage of the individual components in a combination composition or in a combination treatment according to the invention can be determined by a person skilled in the art taken into account the potency of the individual compound, the disease, the age and condition of the patient to be treated etc.

In a further aspect of the invention we have discovered that other alkaline earth metals such gallium and lanthanum, may be provide a structure modifying/chondroprotective effect of pharmaceutical relevance for the prophylactic and/or therapeutic intervention in diseases such as OA and RA.

Accordingly, this invention provides a new method for the treatment, prevention or alleviation of diseases associated with elevated cartilage degradation such as osteoarthritis or rheumatoid arthritis comprising administering an effective amount of a strontium containing compound alone or in combination with one or more agents able to halt or decrease pain and/or structural damage associated with progression of OA or RA.

In particular the present invention relates to the combined administration of a strontium-containing compound such as an inorganic or organic strontium salt with another pharmaceutical compound with structure modifying chondroprotective and/or palliative effects in a patient with an arthritic condition such as RA or OA.

We have discovered that strontium can act in synergy with other analgesic, anti-inflammatory and/or palliative agents by providing a new mechanism of action for treating or preventing pain in the mammalian organism. The method by which ionic strontium mediates the palliative effects has not been completely elucidated, but may partly involve an effect on membrane potential of certain neurons of the CNS by the ability of strontium to bind calcium sensing receptors and calcium gated ion-channels.

Strontium may also exert an effect on peripheral tissues involved in the sensation of pain and transmission of pain signals.

It has previously been disclosed in WO2003FR0003279 that one specific strontium salt, strontium ranelate has the ability to provide relief for gastro-doudenal pain. However, these observations were made with only one specific strontium salt and for only one specific source of pain. We have discovered that a number of other strontium salts, when administered orally to a subject in need thereof, can provide a significant palliative effect, acting by mechanisms independent from existing therapeutic agents such as NSAIDs and opioids. In U.S. Pat. No. 5,866,168 and U.S. Pat. No. 5,851,556 the potential use of certain alkaline earth metal ions, including strontium, in treating unspecified skin pain by being administered topically by i.e. creams and lotions are disclosed. However these references do not disclose the beneficial effects by administering these compounds orally and specifically their potential for acting in synergy with other analgesic or palliative agents. The patents do not disclose any information pertaining to the anti-catabolic effects of strontium on cartilage turnover. In WO 03/028742 it was disclosed that certain strontium compounds may have the ability to ameliorate certain pains originating in soft tissues. In this instance emphasis is put on topical administration although reference is made to the oral administration of a strontium compound for obtaining a palliative effect. However, we have found the especially administering the compound by the oral route provides a systemic palliative effect and we provide specific examples on combinations of drugs applicable of obtaining maximum palliative effect. Furthermore, administration by the oral route is important for evoking the anti catabolic effect of the strontium compounds that we have found. Accordingly, in the present invention we disclose for the first time, that ionic strontium is especially suited for the medical treatment of joint diseases such as OA and RA due to it ability to act in combination on both pain and symptoms of the diseases as well as the underlying catabolic processes of tissue destruction in articular cartilage and subchondral bone.

A central aspect of this invention is the use of an orally administered strontium-containing compound for the alleviation or palliative treatment of acute or chronic conditions involving elevated sensation of pain such as joint disease. The administration may be preceded by, together with or followed by one or more of active substances selected from the group consisting of pallitative agents, disease modifying agents, analgesic agents and anti-inflammatory agents such as those described herein. Such a combination treatment is also of relevance for all other embodiments or aspects of the invention described herein.

In a further aspect of the invention a method of alleviating pain in a mammal including a human is provided, the method comprising administering to the mammal in need thereof a pain alleviating effective amount of a strontium-containing compound for alleviating pain in a mammal in admixture with a pharmaceutically acceptable carrier, diluent, or excipient.

In a further embodiment of the invention, we have found that a strontium containing compound not only enable an improvement in palliative treatment when administered alone, but in particular when administered in combination with another palliative and/or analgesic agent such as a COX-2 specific inhibitor. The beneficial effects of co-administration of a strontium compound applies equally well for therapies with other palliative treatments pharmaceutical drug classes comprising NSAIDs, COX-2 inhibitors, COX-3 inhibitors, combined inhibitors of COX and 5-lipoxygenase, iNOS inhibitors, PAR2 receptor antagonists, neuroleptic agents, opioids, N-acetylcholine receptor agonists, glycine antagonists, vanilloid receptor antagonists, neurokinin antagonists calcitonine gene-related peptide antagonists and Cyclooxygenase (COX)-inhibiting nitric oxide donators (CINOD).

Other aspects of the invention are any one of the above methods of alleviating pain, wherein the pain is one or more of:

osteoarthritic pain,

rheumatoid arthritic pain,

joint pain,

osteoarthritic joint pain,

rheumatoid arthritic joint pain,

acute pain,

acute joint pain,

chronic pain,

chronic joint pain,

inflammatory pain,

inflammatory joint pain,

mechanical pain,

mechanical joint pain, and/or

mediated by EL-6, IIL-6sR, or EL-6 receptor.

Another aspect of the invention is any one of the above methods of alleviating pain other than joint pain, osteoarthritic pain, rheumatoid arthritic pain, and inflammatory joint pain, wherein the pain is pain mediated by IL-6, IL-6sR, or IL-6 receptor.

A still further aspect of the invention is any one of the above methods of alleviating pain, wherein the pain is mediated by a protein or protein and its receptor selected from: oncostatin-M, oncostatin-M and oncostatin-M receptor, leukemia inhibitor factor (“LIF”) and leukemia inhibitor factor receptor (“LIF-R”), interleukin-1 (“IL-1”), and interleukin-1 receptor (“IL1-R”).

Another aspect is any one of the above methods of alleviating pain other than joint pain, osteoarthritic pain, rheumatoid arthritic pain, and inflammatory joint pain, wherein the pain is pain mediated by endothelin.

Another aspect is any one of the above methods of alleviating pain, wherein the pain is associated with a surgical procedure in a patient with a clinical diagnosis of OA, such as orthopedic surgery including but not limited to orthopedic implants used in joint replacement surgery.

### Strontium Salts

A pharmaceutical use according to the invention may be carried out with a number of different strontium salts, either inorganic or organic strontium salts, and furthermore the invention may be carried out with combinations of different strontium salts combined in one pharmaceutical product.

Organic strontium salts have been described, but literature reports of this type of compounds are limited to rather few substance. In these cases the physiochemical properties have been reported to be very similar to the corresponding magnesium, calcium and barium salts. Carboxylic acids can form stable crystalline salts with divalent earth metals such as strontium, and especially di-carboxylic acids are interesting, as they can have a partial chelating effect. Such complexation may be important in biological systems, where the alkaline earth metals, especially calcium and magnesium, play important physiological roles. Hence the divalent metal ions may exist in a complex form in the aqueous environment in biological systems, rather than in a free and un-bound ionic form. Complex formation constants with the alkaline earth metals in aqueous solution are higher for amino acids than for hydroxy-carboxylic acids and the related non-carboxylic acids, which suggest that the amino group may play a role in the complex formation. Generally, the differences in association constants for the various ligands becomes smaller as the radius of the metal increases, and thus the stability of aqueous strontium complexes with di-carboxylic acid is lower than the stability of the comparable complexes with calcium and magnesium.

For a pharmaceutical application of the strontium salts according to the present invention this is very important as it means that strontium salts of dicarboxylic amino acids may be particularly useful.

A strontium salt for a use according to the invention should preferentially be water soluble, and in one embodiment of the present invention, the pH of an aqueous solution of a strontium salt according to the invention has a pH of more than 10. Di-anionic amino-acid salts of strontium, such as strontium aspartate and strontium glutamate but also dicarboxylic anion salts of strontium such as strontium malonate, strontium succinate, strontium pyruvate, strontium fumarate, strontium maleate and strontium oxalate may be especially suited for a pharmaceutical use according to the invention.

Other specific strontium salts which may be used to carry out a medical treatment according to the present inventions will contain an anion with a suitable pharmacologic action such as: strontium L-ascorbate, strontium acetyl-salicylate, strontium salicylate, strontium alendronate, strontium ibandronate, strontium salts of propionic acids such as naproxen, flurbiprofen, fenoprofen, ketoprofen and ibuprofen.

However, this list is not meant to limit the scope of the invention in any way, and a pharmaceutical composition according to the present invention may be manufactured with many different strontium salts comprising both inorganic and organic counter-ions to the strontium ion.

As it appears from the above, the present invention relates to pharmaceutical compositions containing one or more strontium salt alone or in combination with other suitable therapeutically and/or prophylactically active substances for use in the treatment of an acute or chronic condition associated with the sensation of pain.

The inorganic acid for making strontium salts may be selected from the group consisting of boric acid, bromous acid, chloric acid, diphosphoric acid, disulfuric acid, dithionic acid, dithionous acid, fulminic acid, hydrazoic acid, hydrobromic acid, hydrofluoric acid, hydroiodic acid, hydrogen sulfide, hypophosphoric acid, hypophosphorous acid, iodic acid, iodous acid, metaboric acid, metaphosphoric acid, metaphosphorous acid, metasilicic acid, nitrous acid, orthophosphoric acid, orthophosphorous acid, orthosilicic acid, phosphoric acid, phosphinic acid, phosphonic acid, pyrophosphorous acid, selenic acid, sulfonic acid, thiocyanic acid and thiosulfuric acid.

The organic acid may be selected from the group consisting of C₂H₅COOH, C₃H₇COOH, C₄H₉COOH, (COOH)₂, CH₂(COOH)₂, C₂H₄(COOH)₂, C₃H₆(COOH)₂, C₄H₈(COOH)₂, C₅H₁₀(COOH)₂, 2,3,5,6-tetrabromobenzoic acid, 2,3,5,6-tetrachlorobenzoic acid, 2,3,6-tribromobenzoic acid, 2,3,6-trichlorobenzoic acid, 2,4-dichlorobenzoic acid, 2,4-dihydroxybenzoic acid, 2,6-dinitrobenzoic acid, 3,4-dimethoxybenzoic acid, abietic acid, acetoacetic acid, acetonedicarboxylic acid, aconitic acid, acrylic acid, adipic acid, ascorbic acid, aspartic acid (L and D forms), anthranilic acid, arachidic acid, azelaic acid, behenic acid, benzenesulfonic acid, beta-hydroxybutyric acid, benzilic acid, benzoic acid, brassidic acid, carbonic acid, camphoric acid, capric acid, cholic acid, chloroacrylic acid, cinnamic acid, citric acid, citraconic acid, crotonic acid, cyclopentane-1,2-dicarboxylic acid, cyclopentanecarboxylic acid, cystathionine, decanoic acid, erucic acid, ethanesulfonic acid, ethylenediaminetetraacetic acid, folic acid, formic acid, fulvic acid, fumaric acid, gallic acid, glutaconic acid, gluconic acid, glutamic acid (L an D), glutaric acid, gulonic acid, heptanoic acid, hexanoic acid, humic acid, hydroxystearic acid, ibuprofenic acid, isophthalic acid, itaconic acid, lactic acid, lanthionine, lauric acid (dodecanoic acid), levulinic acid, linoleic acid (cis,cis-9,12-octadecadienoic acid), malic acid, m-chlorobenzoic acid, malic acid, maleic acid, malonic acid, melissic acid, mesaconic acid, methacrylic acid, methanesulfonic acid, monochloroacetic acid, myristic acid, (tetradecanoic acid), nonanoic acid, norvaline, octanoic acid, oleic acid (cis-9-octadecenoic acid), ornithine, oxaloacetic acid, oxalic acid, palmitic acid (hexadecanoic acid), p-aminobenzoic acid, p-chlorobenzoic acid, petroselic acid, phenylacetic acid, p-hydroxybenzoic acid, pimelic acid, propiolic acid, phthalic acid, propionic acid, p-tert-butylbenzoic acid, p-toluenesulfonic acid, pyruvic acid, ranelic acid, sarcosine, salicylic acid, sebacic acid, serine, sorbic acid, stearic acid (octadecanoic acid), suberic acid, succinic acid, tartaric acid, terephthalic acid, tetrolic acid, L-threonic acid, thyronine, tricarballylic acid, trichloroacetic acid, trifluoroacetic acid, trimellitic acid, trimesic acid, tyrosine, ulmic acid, valeric acid, vanilic acid and cylohexanecarboxylic acid.

All acids, which FDA has regarded as safe for use in compositions for oral intake, may be used in the present invention.

In one embodiment of the invention, the acid may be a non-chelator of strontium. In yet a further embodiment, the acid may be a monoprotic or a diprotic acid.

In a specific embodiment of the invention, the strontium salt for use according to the invention is water soluble and it may have a water solubility of at least 1 g/l, such as, e.g., at least 5 g/l, at least 10 g/l, at least 20 g/l, at least 30 g/l, at least 40 g/l, at least 50 g/l, at least 60 g/l, at least 70 g/l, at least 80 g/l, at least 90 g/l or about 100 g/l measured at room temperature, i.e. a temperature of 20-25° C.

Specific examples of strontium salts for use according to the invention are strontium malonate, strontium succinate, strontium fumarate, strontium pyrovate, strontium oxalate, strontium ascorbate, strontium aspartate in either L and/or D-form, strontium glutamate in either L- and/or D-form, strontium pyruvate, strontium acetyl salicylate, strontium salicylate, strontium ibuprofenate, strontium tartrate, strontium glutarate, strontium maleate, strontium methanesulfonate, strontium benzenesulfonate and mixtures thereof.

In another embodiment of the invention, the acid may a DMARD such as Doxycycline, Chondroitin Sulfate, Methotrexate, Leflounomide (ARAVA®), azatriopine, salazopyrine, penicillamine, aurothiomalate (gold salt), cyclophosphamide, and azathioprine as well as pharmacologically active derivatives of any of the molecules.

In another embodiment of the invention, the acid may be a bisphosphonate selected from the group consisting of ibandronate, zoledronate, alendronate, risedronate, ethidronate, chlodronate, tiludronate, minodronate, incadronate, olpadronate and pamidronate and pharmacologically active derivatives of any of the molecules.

### Pharmaceutical Formulations

The present invention relates to pharmaceutical compositions comprising an effective amount of a strontium-containing compound according to the invention and a pharmaceutical carrier or diluent as well as potentially other pharmaceutical substances of relevance for the medical intervention in a patient with a joint disease such as OA and RA. Such compositions are preferably in the form of an oral dosage unit or parenteral dosage unit. Especially an oral administration of one or more pharmaceutical compounds according to the invention is preferred. The compounds with which the invention is concerned may also be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch or microcrystalline cellulose, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan mono-oleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or coloring agents.

### DEFINITIONS

As used herein “osteoarthritis” or “OA” means a type of arthritis that is caused by breakdown of cartilage with eventual loss of the cartilage of the joints. The condition may manifest itself in one or only a few joints or it may present as a systemic deterioration of multiple joints. Cartilage is a protein substance that serves as a “cushion” between the bones of the joints. Osteoarthritis is also known as degenerative arthritis or arthrosis. Although OA is not considered an inflammatory disease, there may be both systemic and/or local elevations in inflammatory activity, which may play a role in OA pathogenesis.

As used herein “rheumatoid arthritis” or “RA” means an inflammatory condition where articular cartilage of affected joints is being degraded, by a process where inflammation (localized at the affected joint(s) and/or systemic) takes a prominent role. Levels of several inflammation markers such as C-reactive protein, pro-inflammatory cytokines and certain prostaglandins are elevated in RA. RA may be restricted to a few joints or it may be systemic affecting multiple skeletal sites. The etiology of RA is complex and a number of environmental and genetic factors have been suggested a role in the development of the disease.

As used herein the term ‘Disease modifying anti rheumatic drug’ or DMARD, also known as disease modifying anti-osteoarthritis drug (DMOAD) comprise a heterogeneous group of compounds Doxycycline, Chondroitin Sulfate, hyaluronic acid, Methotrexate, Leflounomide (ARAVA®, Aventis), Dimethylnitrosamine, azatriopine, hydroxychloroqine, cyclosporine, minocycline, salazopyrine, penicillamine, aurothiomalate (gold salt), cyclophosphamide, and azathioprine. Furthermore, some recently introduced biological anti-inflammatory agents can be considered as belonging to the DMARD class as the term is defined in this invention. The pharmaceutical products can function as TNF-α antagonists such as etanercept (Enbrel®, Amgen), adalimumab (Humira® Abott), infliximab (Remicade®, Centocor) or IL-1 receptor antagonists such as the Interleukin-1 receptor antagonist Kineret® (Amgen). Also Osteoprotegrin as well as agonists of this soluble RANK-ligand decoy receptor, such as the monoclonal antibody AMG-162 (Amgen), may be considered a DMARD in the context of the present invention.

For the scope of this invention the class of compounds categorized as non-steroidal antiinflammatory agents (hereinafter NSAID's) comprise molecules such as enolic acis such as piroxicam, tenoxicam and meloxicam, heteroaryl acetic acids such as diclofenac, tolmetin, ketorolac, misoprostol and zomepirac; Indole and indene acetic acids such as indomethacin, mefenamic acid, sulindac and etodolac; Para-amino phenol derivates such as phenacetin and acetaminophen; propionic acids including naproxen, flurbiprofen, fenoprofen, oxaprozin, carprofen, ketoprofen and ibuprofen; fenamates including mefenamic acid, meclofenamate and flufenamic acid; alkanones such as nabumetome; pyrazolones including phenylbutazone, oxyphenbutazone, antipyrine, aminopyrine and kebuzone, salicylates including acetyl salicylate (aspirin), salicylate, salsalate, difunisal, olsalazine, fendosal, sulfasalazine and thiosalicylate, COX-2 inhibitors such as celecoxib (tradename CELEBREX® by G. D. Searle & Co., Skokie, Ill.), valdecoxib (tradename BEXTRA® by Pharmacia & Upjohn Company, North Peapack, N.J.), etoricoxib (tradename ARCOXIA® by Merck & Co., Inc., Whitehouse Station, N.J.), lumiracoxib (tradename PREXIGE® by Novartis AG, Basel, Switzerland), parecoxib, and rofecoxib (tradename VIOXX® by Merck & Co., Inc., Whitehouse Station, N.J.), deracoxib (tradename DERAMAXX® by Novartis AG, Basel, Switzerland) and methylfulfonyl compounds such as sc-558 and sc-58152.

For the purposes of this invention, a selective inhibitor of COX-2 includes a compound, or a pharmaceutically acceptable salt thereof, selected from the group comprising: LAS-34475; UR-8880; ABT-963; Valdecoxib; BMS-347070; Celecoxib; Tilacoxib; (1,1-dimethylheptyl)-6a,7,10,10a-tetrahydro-1-hydroxy-6,6dimethyl-6H-dibenzo[b,d]pyran carboxylic acid (“CT-3”); CV-247; 2(5H)-Furanone, 5,5-dimethyl (1-methylethoxy) [4(methylsulfonyl)phenyl]-(“DFP”); Carprofen (trade name RIMADYLO by Pfizer, Inc., New York, N.Y.); Deracoxib (tradename DERAM [AXX@ by Novartis AG, Basel, Switzerland); Etoricoxib (tradename ARCOXIA@ by MERCK & CO., Inc., Whitehouse Station, N.J.); GW-406381; Tiracoxib; Meloxicam; Nimesulide; 2-(Acetyloxy)benzoic acid, 3-[(nitrooxy)methyl]phenyl ester (“NCX4016”); Lumiracoxib (tradename PREXIGE@ by Novartis AG, Basel, Switzerland); Parecoxib (trade name application pending for DYNASTAT@ by G. D. Searle & Co., Skokie, Ill.); P54 (CAS Reg. No. 130996 0); Rofecoxib (tradename VIOXX@ by MERCK & CO., Inc., Whitehouse Station, N.J.); 2,6-Bis(1,1-dimethylethyl) [(E)-(2-ethyl-1,1-dioxo isothiazolidinylidene)methyl]phenoI (“S-2474”); 5(R)-Thio sulfonamide-3(2H)-benzofuranone (“SVT-2016”); and N-[3-(Formyl-amino) oxo phenoxy-4H benzopyran yl]methanesulfonamide (“T-614”); or a pharmaceutically acceptable salt thereof.

The terra “celecoxib” means the compound named 4-(5-(4-methylphenyl) 3-(trifluoromethyl)-1H-pyrazol-t-yl)-benzenesulfonamide. Celecoxib is a selective cyclooxygenase-2 (“COX-2”) inhibitor currently approved by the FDA for the treatment of osteoarthritis, rheumatoid arthritis, and Polyposis-familial adenomatus. Celecoxib is marketed under the tradename “CELEBREX”.

The term “valdecoxib” means the compound named 4-(5-methyl phenyl4-isoxazolyl)-benzenesulfonamide, which is described in U.S. Pat. Nos. 5,633,272; 5,859,257; and 5,985,902, which are hereby incorporated by reference herein. Valdecoxib has been approved by the FDA for treating osteoarthritis, rheumatoid arthritis, dysmenorrhea, and general pain, and is marketed under the tradename “BEXTRA”.

In addition to the specific examples of COX-2 selective compounds listed above, a great number of selective COX-2 inhibitors are disclosed in the prior art literature and may be used in a pharmaceutical composition according to the present invention. Examples of COX-2 inhibitors are disclosed in, for example, U.S. Pat. Nos. 5,681,842; 5,750,558; 5,756,531; 5,776,984 and in WO 97/41100, WO 98/39330, WO 99/10331, WO 99/10332 and WO 00/24719 assigned to Abbott Laboratories; and in WO 98/50075, WO 00/29022 and WO 00/29023 assigned to Algos Pharmaceutical Corporation; and in WO 99/15205 assigned to Ahnirall Prodesfarma S.A.; and in U.S. Pat. No. 5,980,905 assigned to AMBI Inc.; and in U.S. Pat. No. 5,945,538 assigned to American Cyanamid Company; and in U.S. Pat. Nos. 5,776,967, 5,824,699; 5,830,911 and in WO 98/04527 and WO 98/21195 assigned to American Home Products Corporation; and in WO 98/22442 assigned to Angelini Richerche S.P.A. Societa Consortile; and in U.S. Pat. No. 6,046,191 and in WO 99/18960 and WO 00/00200 assigned to Astra Pharmaceuticals Ltd.; and in U.S. Pat. No. 5,905,089; assigned to Board of Supervisors of Louisiana State University; and in U.S. Pat. Nos. 5,620,999; 5,633,272; 5,643,933, 5,668,161; 5,686,470; 5,696,431; 5,719,163; 5,753,6881; 5,756,530; 5,760,068; 5,859,2571; 5,908,852; 5,935,990; 5,972,986; 5,985,902; 5,990,148; 6,025,353; 6,028,072; 6,136,839 and in WO 94/15932; WO 94/27980; WO 95/11883; WO 95/15315; WO 95/15316; WO 95/15317; WO 95/15318, WO 95/21817; WO 95/30652; WO 95/30656; WO 96/03392; WO 96/03385; WO 96/03387; WO 96/03388; WO 96/09293; WO 96/09304; WO 96/16934; WO 96/25405; WO 96/24584; WO 96/24585; WO 96/36617; WO 96/384181; WO 96/38442; WO 96/41626; WO 96/41645; WO 97/11704; WO 97/27181; WO 97/29776; WO 97/38986; WO 98/06708; WO 98/43649; WO 98/47509; WO 98/47890, WO 98/52937; WO 99/22720; WO 00/23433; WO 00/37107; WO 00/38730; WO 00/38786 and WO 00/53149 assigned to G.D. Searle & Co.; and in WO 96/31509; WO 99/12930; WO 00/26216 and WO 00/52008 assigned to Glaxo Group Limited; and in EP 1 006 114 A1 and in WO 98/46594 assigned to Grelan Pharmaceutical Co. Ltd.; and in WO 97/34882 assigned to Gruppo Farmaceutico Almirall-and in WO 97/03953 assigned to Hafslund Nycomed Pharma AG; and in WO 98/32732 assigned to Hoffmann-La Roche AG; and in U.S. Pat. Nos. 5,945,539; 5,994,381; 6,002,014 and in WO 96/19462; WO 96/19463 and in EP 0 745 596 A1 assigned to Japan Tobacco, Inc.; and in U.S. Pat. Nos. 5,686,460; 5,807,873 and in WO 97/37984; WO 98/05639; WO 98/11080 and WO 99/21585 assigned to Laboratories USPA; and in WO 99/62884 assigned to Laboratories Del Dr. Esteve, S.A.; and in WO 00/08024 assigned to Laboratorios S.A.L.V.A.T., S.A.; and in U.S. Pat. Nos. 5,585,504; 5,840,924; 5,883,267; 5,925,631; 6,001,843; 6,080,876 and in WO 97/44027; WO 97/44028; WO 97/45420; WO 98/03484; WO 98/41511; WO 98/41516; WO 98/43966; WO 99/14194; WO 99/14195; WO 99/23087, WO 99/41224 and WO 00/68215 assigned to Merck Frosst Canada & Co., and in WO 99/59635 assigned to Merck Sharp & Dohme Limited; and in U.S. Pat. No. 5,380,738 assigned to Monsanto Company; and in WO 00/01380 assigned to A. Nattermann & Co.; and in WO 99/61016 assigned to Nippon Shinyaku Co. Ltd.; and in WO 99/33796 assigned to Nissin Food Products Co. Ltd.; and in WO 99/11605 assigned to Novartis AG; and in WO 98/33769 assigned to Nycomed Austria GMBH; and in U.S. Pat. Nos. 6,077,869 and 6,083,969 and in WO 00/51685 assigned to Ortho-McNeil Pharmaceutical, Inc.; and in U.S. Pat. No. 5,783,597 assigned to Ortho Pharmaceutical Corporation; and in WO 98/07714 assigned to Oxis International Inc.; and in WO 00/10993 assigned to Pacific Corporation; and in EP 0 937 722 A1 and in WO 98/50033; WO 99/05104; WO 99/35130 and WO 99/64415 assigned to Pfizer Inc.; and in WO 00/48583 assigned to Pozen Inc.; and in U.S. Pat. No. 5,908,858 assigned to Sankyo Company Limited; and in WO 97/25045 assigned to SmithKline Beecham Corporation; and in U.S. patent No. assigned to Takeda Chemical Industries, Ltd.; and in WO 99/20589 assigned to The University of Sydney; and in U.S. Pat. No. 5,475,021 and WO 00/40087 assigned to Vanderbilt University; and in WO 99/59634 assigned to Wakamoto Pharmaceutical Co. Ltd., the disclosures of each of which are incorporated by reference herein in their entirety.

Any one of the substances listed above or any combinations thereof may be used to carry out the present invention. Furthermore, it follows that a person skilled in the art may devise derivatives of any one of the organic molecules listed above such as, but not limited to, esters, salts, alkylated forms, forms modified by attachment of side-groups selected from the group comprising halogen, alkyl, halogenoalkyl, alkoxy, aryloxy, halogenalkoxy, alkylthio, lower alkylene radical, hydroxyl, nitro, alkylsulfinyl, alkylsulfonyl, sulfamoyl, N-alkylsulfamoyl; aza-, oxa- or thia-lower alkylene radicals, such as 3- or 4-aza-lower alkylene that is unsubstituted or N-substituted by lower alkyl, hydroxy-lower alkyl, lower alkoxy-lower alkyl or by lower alkanoyl, 3- or 4-oxa-lower alkylene or optionally S-oxidised 3- or 4-thia-lower alkylene or another aliphatic group such as a phenyl, thiophenyl, thiophene, fumarate, furan, pyrrole, pyridine, piperidine, imidazole, quinoline, isoquinoline or carbazole group in either unsubstituted form or substituted with one or more lower alkyl or hydroxyl-alky, or amino alkyl groups having from 1 to 7 carbon atoms.

For the purpose of this invention the term ‘opioids’ may be considered to comprise both naturally occurring compounds including endorphins, nociceptin, endomorphins, and synthetically manufactured compounds with the common property of being able to bind opioid receptors in the central nervous system (CNS) as well as in the periphery, thereby providing a substantial palliative effect. Any compound with the ability to bind an opioid receptor with an affinity constant below 10 mM, preferable below 1 mM, more preferably below 0.1 mM or even more preferably below 10 μM can be used to carry out the present invention, but in a preferred embodiment of the invention a selective agonist of the mu-1 receptor is used. Examples of opioids include heroin, fentanyl, morphine, oxycodone, hydrocodone, methadone, buprenorphine, pentazocine, butorphanol, dezocine, nalbuphine, Meperidine, normeperidine, hydromorphone, codeine, levorphanol and tramadol, including any active metabolites thereof.

The invention is further illustrated in the following non-limiting examples. Other aspects and embodiments appear from the appended claims.

### EXAMPLES

### Example 1

### Pharmaceutical Composition Containing Alendronate and a Strontium Compound

Tablet formulation

| Ingredient | Amount (mg)/tablet | |
|---|---|---|
| Alendronate | 10 | mg |
| Strontium malonate | 200 | mg |
| Lactose Ph. Eur. | 100 | mg |
| Corn starch Ph. Eur. (for mixing) | 15 | mg |
| Corn starch Ph. Eur. (for paste) | 15 | mg |
| Magnesium Stearate Ph. Eur. (1%) | 10 | mg |
| Total | 350 | mg |

Alendronate and strontium malonate, lactose and cornstarch (for mixing) are blended to uniformity. The cornstarch for paste is suspended in 200 ml of water and heated with stirring to form a paste. The paste is used to granulate the mixed powders (wet granulation). The wet granules are passed through a number 8 hand screen and dried at 80° C. After drying, the granules are lubricated with 1% magnesium stearate and pressed into a tablet. Such tablets can be administered to a human subject in need thereof, such as an OA or RA patient, from one to two times daily

### Example 2

### Pharmaceutical Composition Containing Methotrexate and a Strontium Compound

Tablet formulation

| Ingredient | Amount (mg)/tablet | |
|---|---|---|
| Methotrexate | 20 | mg |
| Strontium malonate | 200 | mg |
| Lactose Ph. Eur. | 100 | mg |
| Corn starch Ph. Eur. (for mixing) | 15 | mg |
| Corn starch Ph. Eur. (for paste) | 15 | mg |
| Magnesium Stearate Ph. Eur. (1%) | 10 | mg |
| Total | 360 | mg |

The tablets are prepared as described in Example 1.

### Example 3

### Pharmaceutical Composition Containing Naproxen and a Strontium Compound

Tablet formulation

| Ingredient | Amount (mg)/tablet | |
|---|---|---|
| Naproxen | 250 | mg |
| Strontium malonate | 210 | mg |
| Lactose Ph. Eur. | 100 | mg |
| Corn starch Ph. Eur. (for mixing) | 15 | mg |
| Corn starch Ph. Eur. (for paste) | 15 | mg |
| Magnesium Stearate Ph. Eur. (1%) | 10 | mg |
| Total | 500 | mg |

The tablets are prepared as described in Example 3.

### Example 4

### Pharmaceutical Composition Containing Celecoxib and a Strontium Compound

Tablet formulation

| Ingredient | Amount (mg)/tablet | |
|---|---|---|
| Celecoxib | 200 | mg |
| Strontium malonate | 200 | mg |
| Lactose Ph. Eur. | 100 | mg |
| Corn starch Ph. Eur. (for mixing) | 15 | mg |
| Corn starch Ph. Eur. (for paste) | 15 | mg |
| Magnesium Stearate Ph. Eur. (1%) | 10 | mg |
| Total | 540 | mg |

The tablets are prepared as described in Example 1.

### Example 5

### Pharmaceutical Composition Containing 600 mg Strontium Malonate

Tablet formulation

| Ingredient | Amount (mg)/tablet | |
|---|---|---|
| Strontium malonate | 600 | mg |
| Microcrystalline Cellulose Ph. Eur. | 87 | mg |
| Polyvidone Ph. Eur. | 24 | mg |
| Colloidal anhydrous silica Ph. Eur. | 5 | mg |
| Magnesium Stearate Ph. Eur. | 5 | mg |

| | |
|---|---|
| Purified water Ph. Eur. | q.s. |

The following manufacturing procedure is followed for manufacture of approximately 5000 tablets. It follows that the manufacturing procedure may be easily upscaled for preparation of larger batches of tablets. It also follows that different dosage units can be obtained from this recipe simply by using different stamping tools for preparing the tablets.

Strontium malonate (3600 g) and Microcrystalline Cellulose (Avicell, 180 g) is mixed thoroughly in suitable mixing equipment. After mixing the material is filtered through a 1 mm diameter sieve. Over a period of 2 min and under constant mixing, Polyvidone (144 g) and purified water (450 g) are added to the mixture. Additional water may be added if required for obtaining a homogenous granulate. When a homogenous granulate has been obtained, it is placed on trays for drying, and placed in a drying cupboard at 40° C., for 2½-3 hours. The dried granulate is passed through a 1 mm diameter sieve. The Colloidal Anhydrous Silica (23 g) and remaining Microcrystalline Cellulose (Avicell, 284 g) is mixed thoroughly and sieved through a 0.7 mm diameter sieve. The granulate and the silica-cellulose mixture is blended. Magnesium Stearate (23 g) is sieved through a 0.7 mm diameter sieve and premixed with approximately 350 g of the mixture, and when a homogenous mixture has been obtained, the rest of the mixture is added. The mixture is added to a compression tabletting machine, and 721 mg (600 mg strontium malonate) tablets are pressed in cylindrical oblong tablet stamps.

### Example 6

### Assessment of Structure Modifying Effects of Strontium Salts in an Animal Model of Postmenopausal Osteoarthritis

The present study is designed to evaluate the role of strontium in regulating cartilage turnover in an animal model of spontaneous osteoarthritis like joint deterioration (Høegh-Andersen et al Arthritis Res. Ther. 2004, 6: 169-180). The in vivo model is based on the accelerated cartilage loss observed in aged female rats after ovariectomy (OVX), which is a model comparable to the elevation in cartilage turnover observed in women after the menopause resulting in a subsequent increased incidence of osteoarthritis observed in women after the menopause (Mouritzen et al. Annals Rheum Dis. 62: 332-336).

In this experiment rats are OVX treated and subjected to treatments with vehicle alone or with one of four strontium salts (strontium malonate, strontium glutamate, strontium aspartate and strontium ranelate). After 5 weeks treatment the animals are killed and bone and joint tissue removed for histological analysis. Histological analysis of the knee joint is used to assess the pathological changes of the articular cartilage erosions. Furthermore bone and cartilage turnover is assessed by biochemical markers of collagen type I and II degradation (CTX-I and CTX-II).

### Animals and Study Design

The rats used in the experiments are Sprague-Dawley rats, Crl:CD®(SD)IGS.BR obtained from Charles River laboratories, Kisslegg, Germany. The animals are housed with two animals/cage in a room maintained at 20° C. on 12 h light/12 h dark cycles and given food (Altromin 1234, Lage, Germany) and Milli Q water ad libitum.

For the experiment, the rats are maintained in the animal facility for one month after transport from the animal supplier and then divided in two groups subjected to either bilateral ovariectomy using a dorsal approach or a standard sham-operation under general anesthesia induced by Hypnorm-Dormicum (1 part Hypnorm+1 part Dormicum+2 part sterile de-ionized water, dose 0.2 ml/100 g body weight). During the 9 weeks of follow-up, body weight is determined on a weekly basis; urine samples are obtained at baseline and week 2, 4, 6, and 9 after OVX. At study termination, the knees are isolated and kept in 4% formaldehyde until further quantification of surface erosion in the articular cartilage by histological measurements as outlined below.

### Effect of Strontium Salts in OVX Rats

For this purpose, a cohort of seventy-two 6-month old virgin female Sprague-Dawley rats is included. At baseline, body weight is determined and the animals are randomly stratified into six groups with twelve rats in each group. One group is subjected to sham operation and the remaining five groups are ovariectomized as described above. The 5 equal groups receive treatment either with vehicle, strontium glutamate, strontium aspartate, strontium malonate or strontium ranelate according to the scheme listed in table 2.

TABLE 2
Dosing of Strontium compounds in bone efficacy study S01-0401

| | Dose* | Strontium | | | |
|---|---|---|---|---|---|
| Group | (mg/kg) | salt | MW | % Sr | Dose Equivalent |
| Con- | Con- | — | — | — | — |
| trol | trol | | | | |
| B | 500 | Sr-ranelate | 639.6 | 27.4 | 500 mg = 137 mg Sr⁺⁺ |
| | | (*7H₂O) | | | |
| C | 533 | Sr-glutamate | 340.7 | 25.7 | 137 mg Sr⁺⁺ = 533 mg |
| | | (*6H₂O) | | | |
| D | 427 | Sr-aspartate | 272.7 | 32.1 | 137 mg Sr⁺⁺ = 427 mg |
| | | (*3H₂O) | | | |
| E | 325 | Sr-malonate | 207.7 | 42.2 | 137 mg Sr⁺⁺ = 325 mg |
| | | (*1H₂O) | | | |

The strontium salts are given as oral suspension in 0.5% carboxy-methyl-cellulose (CMC) from 4 weeks after the OVX treatment by gavage 5 days a week. Animals are weighed and sampled for spot urine and serum at regular intervals. At study termination, knee joints are prepared for histology as described below.

### Materials and Buffers

All chemicals are analytical grade and purchased from either Sigma (USA) or Merck (Switzerland). Cell culture reagents are obtained from Life Technologies, UK.

### Histology

After careful dissection, the knees are decalcified for 3-4 weeks in 10% formic acid, 2% formaldehyde. The decalcified knee joints are cleaved along the medial collateral ligament into two sections and embedded in paraffin. Coronal sections are then cut in three different depths (0, 250, and 500 μm) from the medial collateral ligament. Each section is stained in Toloudine Blue, and the section that comprises the most loads bearing region is used for measurements. Each knee is blinded and measured separately. In order to simplify evaluation protocols and increase the robustness of the scoring system, a quantitative evaluation on surface erosion is performed as the main parameter of cartilage damage. This approach enables quantifications of erosion in exact numerical values instead of scores relying on the observer. It furthermore relates to a parameter, which is directly relevant to development of OA lesions.

Biomarkers of Bone and Cartilage Degradation

Bone resorption is quantified using an assay, which measures collagen type I C-telopeptide degradation products (CTX-I) using a specific monoclonal antibody in a competitive ELISA form. The assay is performed essentially as described by the supplier (Nordic Bioscience Diagnostics A/S, Herlev, Denmark).

Cartilage turnover is quantified using an immunoassay specific for collagen type II C-telopeptide fragments (CTX-II). The assay is developed for measurement of urine samples, and is performed essentially as described by the supplier (Nordic Bioscience Diagnostics A/S, Herlev, Denmark). The concentration of the CartiLaps ELISA (μg/l) is standardized to the total urine creatinine (mmol/l) (JAFFA method, Hoffman la Roche, Switzerland) giving: concentration/creatinine=μg/mmol.

### Results

### Effects of Strontium Treatment on Cartilage Integrity

Knee joints are excised after termination of the experiments and analyzed by histology by looking at Toloudine Blue stained coronal cross sections showing the femur and tibia condyle. The surface erosion is measured as the percentage of the total articular cartilage surface.

### Bone and Cartilage Turnover

Bone and cartilage turnover is quantified in all rats by measurement in serum of CTX-I and urinary measurement of CTX-II reflecting bone and cartilage turnover respectively. The association between bone and cartilage turnover markers CTX-I and CTX-II is assessed in baseline samples from the three study cohorts.

### Example 7

### Assessment of Palliative Effects of Strontium Salts Administered to OA Patients

The aim of this experiment is to evaluate the palliative effects of strontium given to patients with a clinical diagnosis of mild to moderate OA. The patients are selected to comprise OA patients with a clinical diagnosis of OA at either the hip and/or knee joints with a well defined clinical presentation of the disease. Pain and function of the patients are evaluated with a standardized scoring system (WOMAC score) at the initiation of the study and after 2, 4 and 6 weeks, and the response in the strontium treated patients is compared to the response in a similar placebo treated group.

### Study Protocol and Patients

Briefly described the study cohort consists of patients above 50 years of age (mean about 59 years) with OA of the medial femoro-tibial compartment and/or the hip diagnosed according to the clinical and radiological criteria of the American College of Rheumatology. The patients are recruited at a University Hospital. The severity of their disease corresponds to grade 2 or 3 on the Kellgreen and Lawrence scoring scale, with average disease duration of about 5 years. They are divided in two groups equally sized treated with either 1200 mg strontium malonate daily or placebo for six weeks. The strontium malonate used in the study is a defined pure substance produced according to GLP practice and formulated in tablets as described in example 8. Urine samples are obtained at baseline and after 12 month as second morning void samples after overnight fasting.

The primary outcome measures in the trial are disease symptoms as assessed by the Western Ontario and McMasters Universities osteoarthritis index (WOMAC, VA 3.0 version) performed bi-weekly. As a secondary outcome measure, urine samples are obtained at baseline and after 2 and 6 weeks and measured for the presence of cartilage degradation products using the CartiLaps assay specific for C-telopeptide fragments of articular cartilage derived collagen type II.

### CTX-II Measurements

Urinary levels of collagen type II C-telopeptide fragments are measured by the CartiLaps ELISA assay. The assay uses a highly specific monoclonal antibody MAbF46 specific for a 6-amino acid epitope (EKGPDP) derived from the collagen type II C-telopeptide. The assay is performed essentially as described by the manufacturer (Nordic Bioscience A/S, Herlev, Denmark). All samples are measured in duplicates. All samples from one individual are measured in the same ELISA plate and two control samples are included on each ELISA plate. Average intra- and inter-assay CV is calculated. Three genuine control samples are included on each microtitre plate and if measurements deviates more than ±20% from the predetermined values the plate is re-measured.

The concentration of the CTX-II ELISA (ng/l) is standardized to the total urine creatinine (mmol/l): concentration/creatinine=ng/mmol. Creatinine concentration is measured using a Cobas MIRA analyzed according to the manufactures instructions (Roche Diagnostics, Basel, Switzerland).

### Example 8

### Treatment of OA Patients with Ibuprofen and Strontium Ibuprofenate

The aim of this experiment is to evaluate palliative effects as well as the GI protective effects of strontium in mild to moderate OA patients in two groups of patients treated with either a combination of a strontium compound and naproxen alone. The palliative treatment regiments are given to patients with a clinical diagnosis of mild to moderate OA. The patients are selected to comprise OA patients with a clinical diagnosis of OA at either the hip and/or knee joints with a well defined clinical presentation of the disease. Pain and function of the patients are evaluated with a standardized scoring system (WOMAC score) at the initiation of the study and after 2, 4 and 6 weeks. The presence of gastric irritations, including ulcers is determined by upper endoscopic examinations performed at baseline and at study termination. The response in the treated patients is compared to the response in a similar placebo treated group.

### Study Protocol and Patients

Briefly described the study cohort consists of patients above 50 years of age (mean about 59 years) with OA of the medial femoro-tibial compartment and/or the hip diagnosed according to the clinical and radiological criteria of the American College of Rheumatology. The patients are recruited at a clinic of osteoarthritic rehabilitation. The severity of their disease corresponds to grade 2 or 3 on the Kellgreen and Lawrence scoring scale, with average disease duration of about 5 years. They are divided in two groups equally sized treated with either 200 mg naproxen and 1200 mg strontium malonate or 200 mg naproxen alone for six weeks. Urine samples are obtained at baseline and after 12 month as second morning void samples without dietary restrictions.

The primary outcome measures in the trial are the presence of upper GI damage determined by endoscopic examination. As secondary endpoints the presence of disease symptoms is assessed by the Western Ontario and McMasters Universities osteoarthritis index (WOMAC, VA 3.0 version) performed bi-weekly. As a secondary outcome measures biomarkers of bone and cartilage turnover is measured. For the later purpose, urine samples are obtained at baseline and after 2 and 6 weeks and measured for the presence of cartilage degradation products using the CartiLaps assay specific for C-telopeptide fragments of articular cartilage derived collagen type II, and the urine CrossLaps ELISA (CTX-I) specific for osteoclast generated degradation products of bone matrix type I collagen.

### CTX-II Measurements

Urinary levels of collagen type II C-telopeptide fragments are measured by the CartiLaps ELISA assay. The assay uses a highly specific monoclonal antibody MAbF46 specific for a 6-amino acid epitope (EKGPDP) derived from the collagen type II C-telopeptide. The assay is performed essentially as described by the manufacturer (Nordic Bioscience, Herlev, Denmark). All samples are measured in duplicates. All samples from one individual are measured in the same ELISA plate and two control samples are included on each ELISA plate. Average intra- and inter-assay CV is determined. Three genuine control samples are included on each microtitre plate and if measurements deviate more than ±20% from the predetermined values the plate is re-measured.

The concentration of the CTX-II ELISA (ng/l) is standardized to the total urine creatinine (mmol/l): concentration/creatinine=ng/mmol. Creatinine concentration is measured using a Cobas MIRA analyzed according to the manufactures instructions (Roche Diagnostics, Basel, Switzerland).

Of importance for the present invention, the study demonstrates if the combination strontium and naproxen prevent the occurrence of GI side-effect observed in human subjects when administering naproxen alone.

1. A pharmaceutical composition comprising i) a strontium-containing compound as a first therapeutically and/or prophylactically active substance, and ii) a second therapeutically and/or prophylactically active substance selected from the group consisting of bisphosphonates, glucosamine, palliative agents, analgesic agents, disease modifying anti-rheumatic compounds (DMARDs), selective estrogen receptor modulators (SERMs), aromatase inhibitors, non-steroidal anti-inflammatory agents (NSAIDs), COX-2 inhibitors, COX-3 inhibitors, opioids, inhibitors/antagonists of IL-1, inhibitors/antagonists of TNF-α, inhibitors of matrix metallo-proteinases (MMPs), cathepsin K inhibitors, inhibitors/antagonists of RANK-ligand, statins, glucocorticoids, chondroitin sulphate, NMDA receptor antagonists, inhibitors of interleukin-I converting enzyme, Calcitonin gene related peptide antagonists, glycine antagonists, vanilloid receptor antagonists, inhibitors of inducible nitric oxide synthetase (iNOS), N-acetylcholine receptor agonists, neurokinin antagonists, neuroleptic agents, PAR2 receptor antagonists and anabolic growth factors acting on joint tissue components.

2. The pharmaceutical composition according to claim 1, wherein the second therapeutically and/or prophylactically active substance is a bisphosphonate or a glucosamine.

3. The pharmaceutical composition according to claim 2, wherein the second therapeutically and/or prophylactically active substance is a bisphosphonate selected from the group consisting of ibandronate, zoledronate, alendronate, risedronate, ethidronate, chlodronate, tiludronate, minodronate, incadronate, olpadronate and pamidronate.

4. The pharmaceutical composition according to claim 1, wherein the second therapeutically and/or prophylactically active substance is glucosamine sulphate.

5. The pharmaceutical composition according to claim 1, wherein the second therapeutically and/or prophylactically active substance is selected from the group consisting of heroin, fentanyl, morphine, oxycodone, hydrocodone, methadone, buprenorphine, pentazocine, butorphanol, dezocine, nalbuphine, meperidine, normeperidine, hydromorphone, codeine, levorphanol, tramadol, endorphin, nociceptin, endomorphin, and active metabolites thereof.

6. The pharmaceutical composition according to claim 1, wherein the second therapeutically and/or prophylactically active substance is an NSAID selected from the group consisting of enolic acids, heteroaryl acetic acids; Indole and indene acetic acids; Para-amino phenol derivatives propionic acids; Sulphonanilides; fenamates; alkanones; pyrazolones, salicylates; paracetamol; or a pharmaceutically acceptable salt thereof.

7. The pharmaceutical composition according to claim 1, wherein the second therapeutically and/or prophylactically active substance is a selective COX-2 inhibitor, that has a 10 fold higher affinity for the COX-2 isoenzyme compared to COX-1, the selective COX-2 inhibitor being rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-1-hydroxy-6,6dimethyl-6H-dibenzo[b,d]pyran carboxylic acid (CT-3), 2(5H)-Furanone, 5,5-dimethyl (1-methylethoxy)[4(methylsulfonyl)phenyl]-(DFP); Carprofen (RIMADYLO), (Acetyloxy)-benzoic acid, 3-[(nitrooxy)methyl]phenyl ester (NCX4016), P54 (CAS Reg. No. 130996 0) 2,6-Bis(1,1-dimethylethyl) [(E)-(2-ethyl-1,1-dioxo isothiazolidinylidene)methyl]phenoI (S-2474), 5(R)-Thio sulfonamide-3(2H)-benzofuranone (SVT-2016) N-[3-(Formyl-amino)oxo phenoxy-4H benzopyran yl]methanesulfonamide (“T-614”); or a pharmaceutically acceptable salt thereof.

8. The pharmaceutical composition according to claim 1, wherein the second therapeutically and/or prophylactically active substance is a DMARD selected from the group consisting of doxycycline, chondroitin sulfate, methotrexate, leflounomide (ARAVA®, Aventis), dimethylnitrosamine, azatriopine, hydroxychloroqine, cyclosporine, minocycline, salazopyrine, penicillamine, aurothiomalate (gold salt), cyclophosphamide, azathioprine and pharmacologically active metabolites thereof.

9. The pharmaceutical composition according to claim 1, wherein the second therapeutically and/or prophylactically active substance is a selective estrogen receptor modulator selected from the group consisting of raloxifene, arzoxifene, droloxifene, tamoxifen, 4-hydroxy-tamoxifen, 4′-iodotamoxifen, toremifene, (deaminohydroxy)-toremifene, chlomiphene, levormeloxifene, ormeloxifene, chroman derivatives, coumarin derivatives, idoxifene, nafoxidine, TAT-59, LY-353381, CP-336156, MDL-103323, EM-800, ICI-182, ICI 183,780, ICI 164,384, ICI 183,780, ICI 164,384, diethylstilbesterol, genistein, nafoxidine, nitromifene, moxesterol, diphenol hydrochrysene, erythro-MEA, allenolic acid, equilin-3-sulphate, cyclophenyl, chlorotrianisene, ethamoxytriphetol, lasofoxifene, bazedoxifene, genistein, tibolone, ospemifene, tesmilifene, droloxifene, panomifene, zindoxifene, meproxifene, faslodex, and pharmacologically active metabolites of any of the listed substances.

10. The pharmaceutical composition according to claim 1, wherein the second therapeutically and/or prophylactically active substance is selected from the group consisting of inhibitors of IL-1, a monoclonal antibody against IL-1 and soluble IL-1 receptor derivatives.

11. The pharmaceutical composition according to claim 1, wherein the second therapeutically and/or prophylactically active substance is an inhibitor of interleukin-I converting enzyme (ICE).

12. The pharmaceutical composition according to claim 1, wherein the second therapeutically and/or prophylactically active substance is an inhibitor of TNF-α.

13. The pharmaceutical composition according to claim 1, wherein the second therapeutically and/or prophylactically active substance is selected from the group consisting of OPG and other inhibitors of RANK-ligand.

14. The pharmaceutical composition according to claim 1, wherein the second therapeutically and/or prophylactically active substance is selected from the group consisting of inhibitors of a matrix metalloproteinase aggrecanase, cathepsin K and another protease involved in the catabolic processes of tissue destruction in joint diseases.

15. The pharmaceutical composition according to claim 1, wherein the second therapeutically and/or prophylactically active substance is chondroitin sulphate or keratin sulphate.

16. The pharmaceutical composition according to claim 1, wherein the second therapeutically and/or prophylactically active substance is hyaluronic acid.

17. The pharmaceutical composition according to claim 1, wherein the second therapeutically and/or prophylactically active substance is a glucocorticoid.

18. The pharmaceutical composition according to claim 1, wherein the second therapeutically and/or prophylactically active substance is an endothelin-1 antagonist/inhibitor.

19. The pharmaceutical composition according to claim 1, wherein the second therapeutically and/or prophylactically active substance is an anabolic growth factor derived from bone or cartilage matrix proteins.

20. The pharmaceutical composition according to claim 1, wherein the second therapeutically and/or prophylactically active substance is an anabolic growth factor.

21. The pharmaceutical composition according to claim 1, wherein the second therapeutically and/or prophylactically active substance is a statin.

22. The pharmaceutical composition according to claim 1, wherein the second therapeutically and/or prophylactically active substance is an aromatase inhibitor.

23. The pharmaceutical composition according to claim 1, wherein the second therapeutically and/or prophylactically active substance is a sulphated cyclodextrin.

24. The pharmaceutical composition according to claim 1, wherein the strontium-containing compound is selected from the group of organic strontium salts comprising: strontium malonate, strontium succinate, strontium fumarate, strontium ascorbate, strontium aspartate in either L and/or D-form, strontium glutamate in either L- and/or D-form, strontium pyruvate, strontium tartrate, strontium glutarate, strontium maleate, strontium methanesulfonate, strontium benzenesulfonate and strontium ranelate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-Iminopiperidine, strontium methotrexate, strontium salsalate and strontium sulfasalazinate.

25. The pharmaceutical composition according to claim 1, wherein the strontium-containing compound and the second therapeutically and/or prophylactically active substance are present in a single composition.

26. The pharmaceutical composition according to claim 1, wherein the strontium-containing compound and the second therapeutically and/or prophylactically active substance are present in a kit comprising a first and a second container, the first container comprising the strontium-containing compound and the second container comprising the second therapeutically and/or prophylactically active substance.

27. The pharmaceutical composition according to claim 26 further comprising instructions for simultaneous or sequential use of the first and the second therapeutically and/or prophylactically active substance.

28. The pharmaceutical composition according to claim 1 designed for oral administration.

29-39. (canceled)

40. A method for the treatment of a joint disease selected from the group consisting of OA and RA, the method comprising administering to a subject in need thereof an effective dose of a strontium-containing compound via the oral route.

41. A method for the treatment of a joint disease selected from the group consisting of OA and RA, the method comprising administering to a subject in need thereof an effective dose of a strontium-containing compound via the oral route and an effective dose of a second therapeutically and/or prophylactically active substance selected from the group consisting of bisphosphonates, glucosamine, palliative agents, analgesic agents, disease modifying anti-rheumatic compounds (DMARDs), selective estrogen receptor modulators (SERMs), aromatase inhibitors, non-steroidal anti-inflammatory agents (NSAIDs), COX-2 inhibitors, COX-3 inhibitors, opioids, inhibitors/antagonists of IL-1, inhibitors/antagonists of TNF-α, inhibitors of matrix metallo-proteinases (MMPs), cathepsin K inhibitors, inhibitors/antagonists of RANK-ligand, statins, glucocorticoids, chondroitin sulphate, NMDA receptor antagonists, inhibitors of interleukin-I converting enzyme, Calcitonin gene related peptide antagonists, glycine antagonists, vanilloid receptor antagonists, inhibitors of inducible nitric oxide synthetase (iNOS), N-acetylcholine receptor agonists, neurokinin antagonists, neuroleptic agents, PAR2 receptor antagonists and anabolic growth factors acting on joint tissue components.

42. A method for the treatment of a joint disease selected from the group consisting of OA and RA, the method comprising administering to a subject in need thereof an effective dose of a strontium-containing compound via the oral route and an effective dose of a second therapeutically and/or prophylactically active substance selected from the group consisting of palliative agents, analgesic agents and anti-inflammatory agents.

43. (canceled)

44. The method according to claim 40, wherein the strontium-containing compound is strontium malonate, strontium succinate, strontium fumarate, strontium ascorbate, strontium aspartate in either L and/or D-form, strontium glutamate in either L- and/or D-form, strontium pyruvate, strontium tartrate, strontium glutarate, strontium maleate, strontium methanesulfonate, strontium benzenesulfonate and strontium ranelate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-Iminopiperidine, strontium methotrexate, strontium salsalate or strontium sulfasalazinate.

45. (canceled)

46. The method according to claim 41, wherein the strontium-containing compound and the second therapeutically and/or prophylactically active substance are administered in the form of a single composition.

47. The method according to claim 41, wherein the joint disease is OA and the subject is given a daily dose of ionic strontium corresponding to 100-2000 mg.

48. The method according to claim 41, wherein the joint disease is RA and the subject is given a daily dose of ionic strontium corresponding to 100-2000 mg.

49. The method according to claim 41 where the strontium compound is given in a daily dose of ionic strontium corresponding to 100-2000 mg together with meloxicam administered in a daily dose of 5-20 mg.

50. The method according to claim 41 claims where the strontium compound is given in a daily dose of ionic strontium corresponding to 100-2000 mg together with piroxicam administered in a daily dose of 10-30 mg.

51. The method according to claim 41 where the strontium-containing compound is given in a daily dose of ionic strontium corresponding to 100-2000 mg together with naproxen administered in a daily dose of 500-1500 mg.

52. The method according to claim 41 where the strontium compound is given in a daily dose of ionic strontium corresponding to 100-2000 mg together with dexibuprofen administered in a daily dose of 500-1600 mg.

53. The method according to claim 41 where the strontium compound is given in a daily dose of ionic strontium corresponding to 100-2000 mg together with ibuprofen administered in a daily dose of 1000-3200 mg.

54. The method according to claim 41 where the strontium compound is given in a daily dose of ionic strontium corresponding to 100-2000 mg together with celecoxib administered in a daily dose of 100-200 mg.

55. The method according to claim 41 where the strontium compound is given in a daily dose of ionic strontium corresponding to 100-2000 mg together with salsalate administered in a daily dose of 1000-3000 mg.

56. The pharmaceutical composition according to claim 6, wherein the second therapeutically and/or prophylactically active substance is an enolic acid which is piroxicam, tenoxicam, meloxicam, or a pharmaceutically acceptable salt thereof.

57. The pharmaceutical composition according to claim 6, wherein the second therapeutically and/or prophylactically active substance is a heteroaryl acetic acid which is diclofenac, tolmetin, ketorolac, misoprostol, zomepirac, or a pharmaceutically acceptable salt thereof.

58. The pharmaceutical composition according to claim 6, wherein the second therapeutically and/or prophylactically active substance is an indole or indene acetic acid which is indomethacin, mefenamic acid, sulindac, etodolac, or a pharmaceutically acceptable salt thereof.

59. The pharmaceutical composition according to claim 6, wherein the second therapeutically and/or prophylactically active substance is a para-amino phenol derivative which is phenacetin, acetaminophen, or a pharmaceutically acceptable salt thereof.

60. The pharmaceutical composition according to claim 6, wherein the second therapeutically and/or prophylactically active substance is a propionic acid which is naproxen, flurbiprofen, fenoprofen, oxaprozin, carprofen, ketoprofen, ibuprofen, or a pharmaceutically acceptable salt thereof.

61. The pharmaceutical composition according to claim 6, wherein the second therapeutically and/or prophylactically active substance is Nimesulide or a pharmaceutically acceptable salt thereof.

62. The pharmaceutical composition according to claim 6, wherein the second therapeutically and/or prophylactically active substance is a fenamate which is mefenamic acid, meclofenamate, flufenamic acid, or a pharmaceutically acceptable salt thereof.

63. The pharmaceutical composition according to claim 6, wherein the second therapeutically and/or prophylactically active substance is nabumetome or a pharmaceutically acceptable salt thereof.

64. The pharmaceutical composition according to claim 6, wherein the second therapeutically and/or prophylactically active substance is a pyrazolone which is phenylbutazone, oxyphenbutazone, antipyrine, aminopyrine, kebuzone, or a pharmaceutically acceptable salt thereof.

65. The pharmaceutical composition according to claim 6, wherein the second therapeutically and/or prophylactically active substance is a salicylate which is acetyl salicylate (aspirin), salicylate, salsalate, difunisal, olsalazine, fendosal, sulfasalazine, thiosalicylate, or a pharmaceutically acceptable salt thereof.

66. The pharmaceutical composition according to claim 6, wherein the second therapeutically and/or prophylactically active substance is paracetamol or a pharmaceutically acceptable salt thereof.

67. The pharmaceutical composition according to claim 10, wherein the second therapeutically and/or prophylactically active substance is anakinra.

68. The pharmaceutical composition according to claim 10, wherein the second therapeutically and/or prophylactically active substance is a soluble IL-1 receptor derivative modified by attachment of polyethylene glycol.

69. The pharmaceutical composition according to claim 11, wherein the second therapeutically and/or prophylactically active substance is Pralnacasan.

70. The pharmaceutical composition of claim 12, wherein the inhibitor of TNF-α is etanercept (Enbrel®), adalimumab (Humira®), or infliximab (Remicade®).

71. The pharmaceutical composition of claim 13, wherein the inhibitor of RANK-ligand is monoclonal antibody AMG-162.

72. The pharmaceutical composition according to claim 14, wherein the second therapeutically and/or prophylactically active substance is a matrix metalloproteinase inhibitor which is an inhibitor of MMP-1, MMP-13, or MMP-3.

73. The pharmaceutical composition according to claim 14, wherein the second therapeutically and/or prophylactically active substance is an inhibitor of a protease involved in the catabolic processes of tissue destruction in joint diseases, wherein the joint disease is OA or RA.

74. The pharmaceutical composition according to claim 17, wherein the glucocorticoid is prednisolone, prednisone, methylprednisolone, betamethasone, hydrocortisone, cortisone, triamcinolone, dexamethasone, beclomethasone, budesonide, deoxycortone or fludrocortisone.

75. The pharmaceutical composition according to claim 19, wherein the second therapeutically and/or prophylactically active substance is a segment of or fragment from collagen type I, collagen type II, collagen type IX, collagen type XI, bone sialo protein (BSP), osteonection, osteopontin, osteocalcin (also known as bone GLA protein), cartilage oligomeric matrix protein (COMP), cartilage intermediate layer protein (CILP) or aggrecan.

76. The pharmaceutical composition according to claim 20, wherein the anabolic growth factor is human growth hormone (hGH), parathyroid hormone (PTH), glucagon like peptide-2 (GLP-2), or insulin like growth factor-1 (IGF-1) with or without IGF binding protein 3 (IGFBP-3).

77. The pharmaceutical composition according to claim 21, wherein the statin is nystatin, pravastatin, fluvostatin, atorvastatin and cerivastatin or a therapeutically active derivative thereof.

78. The method of claim 40, wherein the subject is a human.

79. The method of claim 41, wherein the subject is a human.

80. The method of claim 42, wherein the subject is a human.

81. The method according to claim 41, wherein the strontium-containing compound is strontium malonate, strontium succinate, strontium fumarate, strontium ascorbate, strontium aspartate in either L and/or D-form, strontium glutamate in either L- and/or D-form, strontium pyruvate, strontium tartrate, strontium glutarate, strontium maleate, strontium methanesulfonate, strontium benzenesulfonate and strontium ranelate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-Iminopiperidine, strontium methotrexate, strontium salsalate or strontium sulfasalazinate.

82. The method according to claim 42, wherein the strontium-containing compound is strontium malonate, strontium succinate, strontium fumarate, strontium ascorbate, strontium aspartate in either L and/or D-form, strontium glutamate in either L- and/or D-form, strontium pyruvate, strontium tartrate, strontium glutarate, strontium maleate, strontium methanesulfonate, strontium benzenesulfonate and strontium ranelate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-Iminopiperidine, strontium methotrexate, strontium salsalate or strontium sulfasalazinate.
